SEQUENCE LISTING (2 sequences)

SEQ ID NO: 1 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 2 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

(19) United States
(12) Patent Application Publication
(10) Pub. No.: US 2015/0377888 A1
(43) Pub. Date: Dec. 31, 2015

(54) Methods for Predicting and Preventing Metastasis in Triple Negative Breast Cancers

(71) Applicants: INSERM (INSTITUT NATIONAL DE LA SANTÉ ET DE LA RECHERCHE MÉDICALE), Paris (FR); UNIVERSITE DE RENNES I, Rennes (FR); ECOLES DES HAUTES ETUDES EN SANTE PUBLIQUE (EHESP), Rennes (FR); CENTRE EUGENE MARQUIS, Rennes (FR); CHU DE RENNES, Rennes (FR); UNIVERSITE DES ANTILLES ET DE LA GUYANE, Pointe-a-Pitre (FR)

(72) Inventors: Patrick Legembre, Rennes (FR); Marine Malleter, Rennes (FR); Sébastien Tauzin, Rennes (FR); Florence Godey, Rennes (FR); Jean Leveque, Rennes (FR)

(21) Appl. No.: 14/764,326

(22) PCT Filed: Jan. 31, 2014
(86) PCT No.: PCT/EP2014/051884

§ 371 Date: Jul. 29, 2015

Related U.S. Application Data
(60) Provisional application No. 61/772,867, filed on Mar. 5, 2013.

(30) Foreign Application Priority Data
Feb. 1, 2013 (EP) 13305115.1

(51) Int. Cl. G01N 33/574; C07K 16/24

(52) U.S. Cl. CPC G01N 33/57415; C07K 16/241; G01N 2333/70575; C07K 2319/30; C07K 2317/40

(57) ABSTRACT

In particular the present invention relates to a method for predicting the risk of relapse and distant metastasis in a patient suffering from a triple negative breast cancer comprising the step of i) determining the level of soluble CD95L in a blood sample obtained from the patient ii) comparing the level determined at step i) with a predetermined reference value and iii) concluding that the patient will exhibit an increased risk of relapse and distant metastasis when the level determined at step i) is higher than the predetermined reference value or concluding that the patient will exhibit a decreased risk of relapse and distant metastasis when the level determined at step i) is lower than the predetermined reference value. The present invention also relates to a method of preventing metastases in a subject suffering from triple negative breast cancer comprising the steps consisting of i) predicting the risk of relapse and distant metastasis by the method according to the invention and ii) administering the subject with a therapeutically effective amount of a CD95 antagonist when it is concluded at step i) that the subject will exhibit an increased risk of relapse and distant metastasis.

### FIELD OF THE INVENTION

The present invention relates to methods for predicting and preventing metastasis in triple-negative breast cancers.

### BACKGROUND OF THE INVENTION

In cancer, primary tumor cells can acquire the ability to infiltrate surrounding tissues, forming a new tumor called a metastasis, which remains the primary cause of cancer-related mortality (90%). Currently no therapies exist to effectively prevent metastasis (Christofori, 2006). Human breast tumors are heterogeneous, both in their pathology and their molecular profiles. Triple-negative breast cancers (TNBC) are distinguished by negative immunohistochemical staining for estrogen and progesterone receptors and human epidermal growth factor receptor-2 (HER2), and represent 15% of all breast cancers. A recent study categorized breast cancers based on their gene expression profiles as luminal A/B, HER-2, basal-like and normal breast like tumors (Sotiriou and Pusztai, 2009). Based on this analysis, basal-like breast cancer was identified as a close cousin of triple-negative breast cancer (Perou et al., 2000). Triple negative (TN) breast tumors account for a disproportionate number of patient deaths due to their aggressive nature and the lack of effective therapeutic treatment options. Women with TN tumors do not benefit from endocrine therapy or trastuzumab treatment (anti-HER2 mAb) and, currently, no preferred standard form of chemotherapy exists for this type of cancer (Foulkes et al., 2010). Being the most aggressive cancer type with the worst prognosis, TN/basal-like breast cancers exhibit the greatest extent of metastasis compared to other malignant cells (Sorlie et al., 2001).

Thus, gaining insight into the molecular factor(s) and pathways that promote TN breast cancer cell migration may offer substantial therapeutic benefit by revealing potential treatment strategies for preventing metastatic dissemination in TNBC patients.

CD95L (also known as FasL) belongs to the TNF (Tumor Necrosis Factor) family and is the ligand of the “death receptor” CD95 (also known as Fas). In contrast to its ubiquitously expressed receptor, CD95L has been reported to exhibit a restricted expression pattern and is observed primarily at the surface of activated T lymphocytes and NK cells, where it plays a pivotal role in the elimination of infected and transformed cells. CD95L is also expressed on the surface of epithelial cells, macrophages and dendritic cells under inflammatory conditions (Tauzin et al., 2012). CD95L is a transmembrane “cytokine” whose extracellular domain can be cleaved by metalloproteases such as MMP3 (Matsuno et al., 2001), MMP7 (Vargo-Gogola et al., 2002), MMP9 (Kiaei et al., 2007) or ADAM-10 (A Disintegrin And Metalloproteinase 10) (Kirkin et al., 2007; Schulte et al., 2007) to produce a soluble ligand. This soluble ligand was initially described as an inert ligand that competes with its membrane-bound counterpart for binding to CD95, thus acting as an antagonist of the death signal (Schneider et al., 1998; Suda et al., 1997). However, more recent findings demonstrated that metalloprotease-cleaved-CD95L actively participates in aggravating inflammation in chronic inflammatory disorders such as systemic lupus erythematosus (O'Reilly et al., 2009; Tauzin et al., 2011). The CD95/CD95L system has been also endowed with pro-oncogenic functions by promoting proliferation of ovarian and liver cancers (Chen et al., 2010), invasion of glioblastomas (Kleber et al., 2008) and chemotherapy resistance of lung cancers (Bivona et al., 2011) through molecular mechanisms that remain to be elucidated.

From a molecular standpoint, the binding of membrane-bound CD95L (m-CD95L) to CD95 leads to recruitment of the adaptor protein Fas-associated death domain protein (FADD) at the CD95 intracellular region called the death domain (DD). In turn, FADD binds and aggregates caspases 8 and 10. This CD95/FADD/caspase complex, called the Death-Inducing Signaling Complex (DISC) (Kischkel et al., 1995), plays a pivotal role in the initiation of the apoptotic signal. In contrast, metalloprotease-cleaved CD95L fails to induce DISC formation and instead promotes the formation of an atypical complex that we have designated MISC, for Motility-Inducing Signaling Complex (Tauzin et al., 2011). This non-apoptotic complex contains the src kinase c-yes and results in the induction of a calcium (Ca₂₊)/PI3K signaling pathway that promotes the endothelial transmigration of T cells (Tauzin et al., 2011). The mechanistic link that connects CD95 engagement by cl-CD95L to the activation of PI3K remains unknown.

### SUMMARY OF THE INVENTION

The present invention relates to methods for predicting the risk of relapse and distant metastasis in a patient suffering from a triple negative breast cancer. The present invention also relates to methods for treating triple negative breast cancer in a patient in need thereof.

### DETAILED DESCRIPTION OF THE INVENTION

Considered as an apoptotic factor, CD95L is expressed by immune cells to eliminate malignant cells. After cleavage by metalloproteases, this protein is released in blood. Although high amounts of serum CD95L have been detected in cancers, whether or not this cytokine exerts a role in the pathogenesis remains unknown. We show that the amount of serum CD95L is increased in patients with triple negative breast cancer (TNBC) as compared to non-TNBC and is associated with risk of relapse. Analysis of breast tumor architecture reveals that CD95L is expressed on endothelial cells covering blood vessels and after cleavage by metalloprotease, it implements a pro-motile c-yes/EGFR/Ca²⁺/PI3K signaling pathway in TNBC cells. Overall, these findings unveil the prometastatic role of CD95L in breast cancers.

In cancer, metastasis remains the primary cause of mortality and no therapies exist to prevent it. Breast cancers correspond to a heterogeneous pathology where it is crucial to identify prognostic markers to reliably select high and low risk subsets of patients for the likelihood of distant metastasis in order to adapt therapies. We demonstrate that the serum concentration of CD95L is increased in TNBC as compared to non-TNBC patients and predicts risk of distant metastasis. The pro-metastatic role of CD95L was molecularly confirmed as TNBC cells exposed to CD95L undergo an ordered sequence of events—NADPH-driven ROS generation, c-yes activation, EGFR recruitment and opening of Ca²⁺ channel Orai1—that leads to activation of PI3K signal and cell migration.

Predictive Methods:

Accordingly, an aspect of the present invention relates to a method for predicting the risk of relapse and distant metastasis in a patient suffering from a triple negative breast cancer comprising the step of i) determining the level of soluble CD95L in a blood sample obtained from the patient ii) comparing the level determined at step i) with a predetermined reference value and iii) concluding that the patient will exhibit an increased risk of relapse and distant metastasis when the level determined at step i) is higher than the predetermined reference value or concluding that the patient will exhibit a decreased risk of relapse and distant metastasis when the level determined at step i) is lower than the predetermined reference value.

The present invention also relates to a method for predicting the disease-free survival in a patient suffering from a triple negative breast cancer comprising the step of i) determining the level of soluble CD95L in a blood sample obtained from the patient ii) comparing the level determined at step i) with a predetermined reference value and iii) concluding that the patient has a poor prognosis when the level determined at step i) is higher than the predetermined reference value or concluding that the patient has a good prognosis when the level determined at step i) is lower than the predetermined reference value.

As used herein the expression “Triple negative breast cancer” has its general meaning in the art and means that said breast cancer lacks receptors for the hormones estrogen (ER-negative) and progesterone (PR-negative), and for the protein HER2.

By “blood sample” is meant a volume of whole blood or fraction thereof, eg, serum, plasma, etc.

As used herein, the term “CD95” has its general meaning in the art and refers to CD95, the receptor present on the surface of mammalian cells, which has been originally shown to have the capacity to induce apoptosis upon binding of the trimeric form of its cognate ligand, CD95L (Krammer, P. H. (2000). CD95's deadly mission in the immune system. Nature 407, 789-795). CD95 is also known as FasR or Apo-1. An exemplary amino acid sequence of CD95 is shown as SEQ ID NO:1 (UniProtKB/Swiss-Prot accession number: P25445).

```
SEQ ID NO: 1
                10 20 30 40 50 60
MLGIWTLLPL VLTSVARLSS KSVNAQVTDI NSKGLELRKT VTTVETQNLE GLHHDGQFCH
                70 80 90 100 110 120
KPCPPGERKA RDCTVNGDEP DCVPCQEGKE YTDKAHFSSK CRRCRLCDEG HGLEVEINCT
              130 140 150 160 170 180
RTQNTKCRCK PNFFCNSTVC EHCDPCTKCE HGIIKECTLT SNTKCKEEGS RSNLGWLCLL
              190 200 210 220 230 240
LLPIPLIVWV KRKEVQKTCR KHRKENQGSH ESPTLNPETV AINLSDVDLS KYITTIAGVM
              250 260 270 280 290 300
TLSQVKGFVR KNGVNEAKID EIKNDNVQDT AEQKVQLLRN WHQLHGKKEA YDTLIKDLKK
              310 320 330
ANLCTLAEKI QTIILKDITS DSENSNFRNE IQSLV
```

As used herein the term CD95L has its general meaning in the art ant refers to the cognate ligand of CD95 that is a transmembrane protein.

As used herein the term “soluble CD95L” has its general meaning in the art and refers to the soluble ligand produced by the cleavage of the transmembrane CD95L (also known as FasL) (Matsuno et al., 2001; Vargo-Gogola et al., 2002; Kiaei et al., 2007; Kirkin et al., 2007; or Schulte et al., 2007). The term “serum CD95L”, “soluble CD95L”, “metalloprotease-cleaved CD95L” and “cl-CD95L” have the same meaning along the specification. An exemplary amino acid sequence of CD9L5 is shown as SEQ ID NO:2 (UniProtKB/Swiss-Prot accession number: P48023).

```
SEQ ID NO: 2:
                10 20 30 40 50 60
MQQPFNYPYP QIYWVDSSAS SPWAPPGTVL PCPTSVPRRP GQRRPPPPPP PPPLPPPPPP
                70 80 90 100 110 120
PPLPPLPLPP LKKRGNHSTG LCLLVMFFMV LVALVGLGLG MFQLFHLQKE LAELRESTSQ
              130 140 150 160 170 180
MHTASSLEKQ IGHPSPPPEK KELRKVAHLT GKSNSRSMPL EWEDTYGIVL LSGVKYKKGG
              190 200 210 220 230 240
LVINETGLYF VYSKVYFRGQ SCNNLPLSHK VYMRNSKYPQ DLVMMEGKMM SYCTTGQMWA
              250 260 270 280
RSSYLGAVFN LTSADHLYVN VSELSLVNFE ESQTFFGLYK L
```

The predetermined reference value may be determined by any well known method in the art. For example, the predetermined reference value may be typically determined by carrying out a method comprising the steps of

a) providing a collection of blood samples from triple negative breast cancer patients;

b) providing, for each blood sample provided at step a), information relating to the actual clinical outcome for the corresponding triple negative breast cancer patient (i.e. risk of relapse and distant metastasis, the duration of the disease-free survival (DFS) and/or the overall survival (OS));

c) providing a serial of arbitrary quantification values;

d) determining the level of soluble CD95L for each blood sample contained in the collection provided at step a);

e) classifying said blood samples in two groups for one specific arbitrary quantification value provided at step c), respectively: (i) a first group comprising blood samples that exhibit a quantification value for level that is lower than the said arbitrary quantification value contained in the said serial of quantification values; (ii) a second group comprising blood samples that exhibit a quantification value for said level that is higher than the said arbitrary quantification value contained in the said serial of quantification values; whereby two groups of blood samples are obtained for the said specific quantification value, wherein the blood samples of each group are separately enumerated;

f) calculating the statistical significance between (i) the quantification value obtained at step e) and (ii) the actual clinical outcome of the patients from which blood samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the level of soluble CD95L has been assessed for 100 blood samples of 100 patients. The 100 samples are ranked according to the level of soluble CD95L. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the level of soluble CD95L corresponding to the boundary between both subsets for which the p value is minimum is considered as the predetermined reference value. It should be noted that the predetermined reference value is not necessarily the median value of levels of soluble CD95L.

The setting of a single “cut-off” value thus allows discrimination between an increased risk of relapse and distant metastasis and a decreased risk of relapse and distant metastasis (or a poor and a good prognosis with respect to DFS and OS for a patient). Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided.

Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above.

For example, according to this specific embodiment of a “cut-off” value, the outcome can be determined by comparing the level of soluble CD95L with the range of values which are identified. In certain embodiments, a cut-off value thus consists of a range of quantification values, e.g. centered on the quantification value for which the highest statistical significance value is found (e.g. generally the minimum P value which is found). For example, on a hypothetical scale of 1 to 10, if the ideal cut-off value (the value with the highest statistical significance) is 5, a suitable (exemplary) range may be from 4-6. Therefore, a patient may be assessed by comparing values obtained by measuring the level of soluble CD95L, where values greater than 5 reveal an increased risk of relapse and distant metastasis (or a poor prognosis) and values less than 5 reveal a decreased risk of relapse and distant metastasis (or a good prognosis). In a another embodiment, a patient may be assessed by comparing values obtained by measuring the level of soluble CD95L and comparing the values on a scale, where values above the range of 4-6 indicate an increased risk of relapse and distant metastasis (or a poor prognosis) and values below the range of 4-6 indicate a decreased risk of relapse and distant metastasis (or a good prognosis), with values falling within the range of 4-6 indicating an intermediate occurrence (or prognosis).

Typically, the predetermined reference value may be 80 pg/ml or 120 pg/ml (see FIG. 1).

According to the invention, the measure of the level of soluble CD95L can be performed by a variety of techniques. Typically, the methods may comprise contacting the sample with a binding partner capable of selectively interacting with soluble CD95L in the sample. In some aspects, the binding partners are antibodies, such as, for example, monoclonal antibodies or even aptamers.

The aforementioned assays generally involve the binding of the partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The level of soluble CD95L may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

An exemplary biochemical test for identifying specific proteins employs a standardized test format, such as ELISA test, although the information provided herein may apply to the development of other biochemical or diagnostic tests and is not limited to the development of an ELISA test (see, e.g., Molecular Immunology: A Textbook, edited by Atassi et al. Marcel Dekker Inc., New York and Basel 1984, for a description of ELISA tests). It is understood that commercial assay enzyme-linked immunosorbant assay (ELISA) kits for various plasma constituents are available. Therefore ELISA method can be used, wherein the wells of a microtiter plate are coated with a set of antibodies which recognize soluble CD95L. A sample containing or suspected of containing soluble CD95L is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Measuring the level of soluble CD95L (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e.g., retention time, for that compound and measured using standard techniques.

Alternatively, the separated compounds may be detected and measured by, for example, a mass spectrometer.

Typically, levels of immunoreactive soluble CD95L in a sample may be measured by an immunometric assay on the basis of a double-antibody “sandwich” technique, with a monoclonal antibody specific for soluble CD95L (Cayman Chemical Company, Ann Arbor, Mich.). According to said embodiment, said means for measuring soluble CD95L level are for example i) a soluble CD95L buffer, ii) a monoclonal antibody that interacts specifically with soluble CD95L, iii) an enzyme-conjugated antibody specific for soluble CD95L and a predetermined reference value of soluble CD95L.

Kits:

A further object of the invention relates to a kit for performing the above described method, said kit comprising means for measuring the level of soluble CD95L in the blood sample obtained from the patient.

In a particular embodiment, said means for measuring the level of soluble CD95L is an antibody that interacts specifically with soluble CD95L. In another embodiment, said means for measuring the level of soluble CD95L may be an aptamer or any other binding partner that specifically recognizes soluble CD95L.

Said binding partner can be tagged for an easier detection. It may or may not be immobilized on a substrate surface (e.g., beads, array, and the like). For example, an inventive kit may include an array for predicting the risk of having a cardiovascular event as provided herein. Alternatively, a substrate surface (e.g. membrane) may be included in an inventive kit for immobilization of the binding partner (e.g., via gel electrophoresis and transfer to membrane).

In addition, a kit of the invention generally also comprises at least one reagent for the detection of a complex between binding partner included in the kit and biomarker of the invention.

Depending on the procedure, the kit may further comprise one or more of: extraction buffer and/or reagents, western blotting buffer and/or reagents, and detection means. Protocols for using these buffers and reagents for performing different steps of the procedure may be included in the kit.

The different reagents included in a kit of the invention may be supplied in a solid (e.g. lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit comprises instructions for using its components for the prediction of a cardiovascular event in a patient according to a method of the invention. Instructions for using the kit according to methods of the invention may comprise instructions for processing the biological sample obtained from the patient and/or for performing the test, or instructions for interpreting the results. A kit may also contain a notice in the form prescribed by a governmental agency regulating the manufacture, use or sale of pharmaceuticals or biological products.

Methods of Treating:

The present invention also relates to a CD95 antagonist for use in the treatment of triple negative breast cancer in a subject in need thereof.

The present invention also relates to a CD95 antagonist for use in the prevention of metastases in a subject suffering from a triple negative breast cancer.

The present invention also relates to a method of preventing metastases in a subject suffering from triple negative breast cancer comprising the steps consisting of i) method for predicting the risk of relapse and distant metastasis by the method according to the invention and ii) administering the subject with a therapeutically effective amount of a CD95 antagonist when it is concluded at step i) that the subject will exhibit an increased risk of relapse and distant metastasis (i.e. the level of soluble CD95L is higher than the predetermined reference value).

The term “CD95 antagonist” means any molecule that attenuates signal transduction mediated by the binding of CD95 to the soluble CD95L. In particular the CD95 antagonist is a molecule that inhibits, reduces, abolishes or otherwise reduces the formation of Motility-Inducing Signaling Complex induced by the soluble CD95L. In other terms the CD95 antagonist is a molecule that inhibits, reduces, abolishes or otherwise reduces the pro-motile c-yes/EGFR/Ca²⁺/PI3K signaling pathway triggered by the soluble CD95L in TNBC cells. Such inhibition may result where: (i) the CD95 antagonist of the invention binds to a CD95 without triggering signal transduction, to reduce or block signal transduction mediated by soluble CD95L; (ii) the CD95 antagonist binds to the soluble CD95L, preventing its binding to CD95; (iii) the CD95 antagonist binds to, or otherwise inhibits the activity of, a molecule that is part of a regulatory chain that, when not inhibited, has the result of stimulating or otherwise facilitating CD95 signal transduction mediated by soluble CD95L; or (iv) the CD95 antagonist inhibits CD95 expression or CD95L expression, especially by reducing or abolishing expression of one or more genes encoding CD95 or CD95L.

Typically, the CD95 antagonist includes but is not limited to an antibody, a small organic molecule, a polypeptide and an aptamer.

In one embodiment, the agent is an antibody. The invention embraces antibodies or fragments of antibodies. Typically the antibodies of the invention have the ability to block the interaction between soluble CD95L and CD95 or have the ability to block the induction of the signaling pathway mediated by soluble CD95L (e.g. by inhibiting the oligomerisation of CD95). The antibodies may have specificity to soluble CD95L or CD95.

In one embodiment, the antibodies or fragment of antibodies are directed to all or a portion of the extracellular domain of CD95. In one embodiment, the antibodies or fragment of antibodies are directed to an extracellular domain of CD95. More particularly this invention provides an antibody or portion thereof capable of inhibiting binding of CD95 to soluble CD95L, which antibody binds to an epitope located within a region of CD95, which region of CD95 binds to soluble CD95L. Even more particularly, the invention provides an antibody or portion thereof capable of binding to an epitope located within a region of CD95, which region of CD95 is involved the oligomerisation of the receptor. Typically, the antibody binds to the cysteine-rich domain 1 of CD95 which is called the pre-ligand assembly domain (PLAD) (Edmond V, Ghali B, Penna A, Taupin J L, Daburon S, Moreau J F, Legembre P. Precise mapping of the CD95 pre-ligand assembly domain. PLoS One. 2012; 7(9):e46236. doi: 10.1371/journal.pone.0046236. Epub 2012 Sep. 25.). In particular the antibody of the invention binds to the regions delimitated between the amino acid at position 43 and the amino acid at position 66.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of soluble CD95L, or CD95. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant soluble CD95L may be provided by expression with recombinant cell lines. CD95 may be provided in the form of human cells expressing CD95 at their surface. Recombinant forms of CD95 or soluble CD95L may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated a Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In one embodiment of the agents described herein, the agent is a polypeptide. In a particular embodiment the polypeptide is a functional equivalent of CD95. As used herein, a “functional equivalent of CD95 is a compound which is capable of binding to soluble CD95L, thereby preventing its interaction with CD95. The term “functional equivalent” includes fragments, mutants, and muteins of CD95. The term “functionally equivalent” thus includes any equivalent of CD95 obtained by altering the amino acid sequence, for example by one or more amino acid deletions, substitutions or additions such that the protein analogue retains the ability to bind to soluble CD95L. Amino acid substitutions may be made, for example, by point mutation of the DNA encoding the amino acid sequence. Functional equivalents include molecules that bind soluble CD95L and comprise all or a portion of the extracellular domains of CD95.

The functional equivalents include soluble forms of the CD95. A suitable soluble form of these proteins, or functional equivalents thereof, might comprise, for example, a truncated form of the protein from which the transmembrane domain has been removed by chemical, proteolytic or recombinant methods.

Preferably, the functional equivalent is at least 80% homologous to the corresponding protein. In a preferred embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of CD95 that binds to soluble CD95L. Accordingly the present invention provides a polypeptide capable of inhibiting binding of CD95 to soluble CD95L, which polypeptide comprises consecutive amino acids having a sequence which corresponds to the sequence of at least a portion of an extracellular domain of CD95, which portion binds to soluble CD95L. In one embodiment, the polypeptide corresponds to an extracellular domain of CD95.

Functionally equivalent fragments may belong to the same protein family as the human CD95 identified herein. By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

The polypeptides of the invention may be produced by any suitable means, as will be apparent to those of skill in the art. In order to produce sufficient amounts of CD95 or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In still another embodiment, the polypeptides of the invention may be fused to a heterologous polypeptide (i.e. polypeptide derived from an unrelated protein, for example, from an immunoglobulin protein).

As used herein, the terms “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature. Although the reading frame is thus made continuous throughout the fused segments, the segments may be physically or spatially separated by, for example, in-frame linker sequence.

As used herein, the term “fusion protein” means a protein comprising a first polypeptide linearly connected, via peptide bonds, to a second, polypeptide.

As used herein, the term “CD95 fusion protein” refers to a polypeptide that is a functional equivalent of CD95 fused to heterologous polypeptide. The CD95 fusion protein will generally share at least one biological property in common with the CD95 polypeptide (as described above). An example of a CD95 fusion protein is a CD95 immunoadhesin.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The immunoglobulin sequence preferably, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In one embodiment, the Fc region is a native sequence Fc region. In another embodiment, the Fc region is a variant Fc region. In still another embodiment, the Fc region is a functional Fc region. The CD95 portion and the immunoglobulin sequence portion of the CD95 immunoadhesin may be linked by a minimal linker. The immunoglobulin sequence preferably, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but preferably IgG1 or IgG3.

As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

Another example of a CD95 fusion protein is a fusion of the CD95 polypeptide with human serum albumin-binding domain antibodies (AlbudAbs) according to the AlbudAb™ Technology Platform as described in Konterman et al. 2012 AlbudAb™ Technology Platform—Versatile Albumin Binding Domains for the Development of Therapeutics with Tunable Half-Lives

Typically a CD95 fusion according to the invention may be APG101 which is developed by Apogenix™. APG101 is a fully human fusion protein consisting of the extracellular domain of the CD95 receptor and the Fc domain of an IgG antibody.

In one embodiment, the agent is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

In a particular embodiment the CD95 antagonist is an inhibitor of CD95 expression (or CD95L expression).

An “inhibitor of expression” refers to a natural or synthetic compound that has a biological effect to inhibit the expression of a gene. Therefore, an “inhibitor of CD95 expression” denotes a natural or synthetic compound that has a biological effect to inhibit the expression of CD95 gene.

In a preferred embodiment of the invention, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of CD95 mRNA by binding thereto and thus preventing protein translation or increasing mRNA degradation, thus decreasing the level of CD95, and thus activity, in a cell. For example, antisense oligonucleotides of at least about 15 bases and complementary to unique regions of the mRNA transcript sequence encoding CD95 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CD95 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N. J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

By a “therapeutically effective amount” of CD95 antagonist as above described is meant a sufficient amount of the CD95 antagonist. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The CD95 antagonist of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The CD95 antagonist of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The CD95 antagonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The present invention also relates to a method of preventing metastases in a subject suffering from triple negative breast cancer comprising the steps consisting of i) predicting the risk of relapse and distant metastasis by performing the method according to the invention and ii) administering the subject with a therapeutically effective amount of a EGFR antagonist when it is concluded at step i) that the subject will exhibit an increased risk of relapse and distant metastasis (i.e. the level of soluble CD95L is higher than the predetermined reference value).

The present invention also relates to a method of preventing metastases in a subject suffering from triple negative breast cancer comprising the steps consisting of i) predicting the risk of relapse and distant metastasis by performing the method according to the invention and ii) administering the subject with a therapeutically effective amount of a PI3K inhibitor when it is concluded at step i) that the subject will exhibit an increased risk of relapse and distant metastasis (i.e. the level of soluble CD95L is higher than the predetermined reference value). Typically, the PIK3 inhibitor is a PIK3alpha and/or PI3Kbeta inhibitor.

The invention will be further illustrated by the following FIGURES and examples. However, these examples and FIGURES should not be interpreted in any way as limiting the scope of the present invention.

### FIGURES

FIG. 1. Soluble CD95L is increased in TN breast cancer and is associated with metastatic dissemination. A. Soluble CD95L in the serum of breast cancer patients diagnosed with TNBC or non-TNBC was measured by ELISA (n indicates the number of patients per group). ***, ** indicates P≦0.0001 and 0.01 by two-tailed student's t test. B. Kaplan-Meier Relapse-free survival curves comparing patients exhibiting concentrations of CD95L higher (thick line) or lower (dotted line) than 80 pg/ml. C. Upper panel: Kaplan-Meier analysis of distant metastasis among patients with concentrations of CD95L higher (thick line) or lower (dotted line) than 80 pg/ml. Lower panel: Kaplan-Meier analysis of distant metastasis among patients exhibiting concentrations of CD95L higher (thick line) or lower (dotted line) than 120 pg/ml.

### EXAMPLE

### Naturally-Processed CD95L is a Prometastatic Factor in Triple Negative Breast Cancers

Material & Methods

Ethics Statement.

All clinical investigation was conducted in accordance with the principles outlined in the Declaration of Helsinki. Blood was sampled from patients diagnosed with breast cancer after written consent was obtained from each individual. This study was approved by the institutional review board at the Centre Hospitalier Universitaire de Nantes.

Statistical Analysis.

Comparisons between groups were done by Pearson Chi² test (or Fisher exact test if necessary) for qualitative parameters and by ANOVA or Student t test (Kruskal-Wallis or Mann-Whitney test if necessary) for continuous parameters. Metastasis-free survival was calculated from the date of the diagnosis to the date of the first metastasis or last follow-up if no metastatic relapse. Survival data were available for 142 patients. Survival curves were calculated by means of Kaplan-Meier method and groups were compared by means of log rank test. All analyses were done with Stata10.1 special edition (Statacorp, Texas USA). All tests were two-sided with p significant <5%.

Cell Lines and shRNAmir Lentiviral Transduction.

The human breast adenocarcinoma cell lines MDA-MB-231, MDA-MB-468, Hs578T, MCF7, T47D, and ZR75-1 were maintained in DMEM supplemented with 8% v/v heat-inactivated FCS and 2 mM L-glutamine at 37° C. in a 5% CO₂ incubator. Silencing experiments were performed by lentiviral transduction of breast cancer cell lines using validated shRNAmir-pGIPZ vectors (OpenBiosystems, Thermo Scientific, Waltham, Mass., USA) for c-yes (RHS4430-98843955, -99161516, -98843955), Orai1 (RHS4430-98715881, -101067842), p110α (RHS4430-99882045 and RHS4531-NM_{—}006218), p110β (RHS4430-101074610 and RHS4430-101519729) EGF-R (RHS4430-99291735, RHS4430-98895495 and RHS4430-101517905) and p22phox (RHS4430-98520051, RHS4430-98842095 and RHS4430-101071465) or a scrambled shRNAmir vector as a negative control. To improve the percentage of transduced breast cancer cells, living cells were harvested 72 hours after transduction and green cells (pGIPZ encodes GFP) were sorted by flow cytometry using a FACSAria (BD Bioscience).

Cleaved-CD95L Production.

293 cells maintained in 8% FCS-containing medium were transfected using the calcium/phosphate precipitation method with 3 μg of empty plasmid or wild-type CD95L-containing vector. At 16 hours after transfection, the medium was replaced with OPTI-MEM supplemented with 2 mM L-glutamine. Five days later, media containing cleaved CD95L and exosome-bound full length CD95L were harvested. Dead cells and debris were removed by two rounds of centrifugation (4500 rpm/15 min), and exosomes were eliminated by ultracentrifugation (100000×g/2 hours). Supernatants containing cleaved CD95L were kept at 4° C.

Antibodies, Plasmids and Other Reagents.

BTP2, LY294002, BAPTA-AM ([1,2-bis-(o-Aminophenoxy)ethane-N,N,N′,N′-tetraacetic Acid Tetra-(acetoxymethyl) Ester]) and zVAD-fmk (carbobenzoxy-valyl-alanyl-aspartyl-[O-methyl]-fluoromethylketone) were obtained from Calbiochem (Merck Chemicals Ltd., Nottingham, UK). DPX mountant, anti-β-actin, anti-tubulin, DAPI, diphenyleneiodonium (DPI), apocynin, N-acetyl L-cysteine (NAC) and anti-c-yes were purchased from Sigma-Aldrich (L'Isle-d'Abeau-Chesnes, France). Fura-2-PE3/AM and Fluo8-AM were from Tefflabs (Euromedex, Mundolsheim, France). Anti-human CD31 and D2-40, H2FDA (dihydrofluorescein) was from Invitrogen (Saint Aubin, France). Erlotinib, anti-p22Phox, anti-duox1 and anti-nox4 were purchased from Santa Cruz Biotechnology (Santa Cruz, Calif., USA). Cetuximab was obtained from the Centre Hospitalier de Rennes (Dr. Florence Godey, France). Anti-caspase-8 (C15) was purchased from Axxora (Coger S. A., Paris, France). Anti-human CD95 mAb (DX2) and CD95L mAb (G247-4) were from BD Biosciences (Le Pont de Claix, France). Anti-human Orai1, Anti-Akt, anti-phosphoS473 Aid (Akt-^{P473}), anti-phospho Src family members (P-Src), anti-p110α, anti-p110β, anti-EGF-R, anti-phosphoY845 EGFR (EGFR^{Y845}) and anti-phosphoY416 Src antibodies were from Cell Signaling Technology (Boston, Mass., USA). Anti-nox1, anti-nox2, anti-nox3, anti-nox5 were purchased from Abcam (Abcam, Paris, France).

Immunohistochemistry.

Hollande-fixed tumor tissues from breast cancers (non-triple-negative and triple-negative patients) and benign breast diseases (two patients per immunohistochemical staining) were embedded in paraffin and cut into 3 μm sections. For CD95L staining, tissue sections were deparaffinized in xylene for 3 min and rehydrated via graded alcohol treatment. Protein cross-links formed by formalin fixation were reversed (Target Retrieval Solution, pH 9) and endogenous peroxidase was blocked using 3% w/v hydrogen peroxide in methanol for 15 min. Slides were incubated in 5% BSA for 30 min at RT and then stained overnight with mouse anti-CD95L (1:300) or mouse anti-CD31 (1:100) or mouse anti-D240 (1:100) at 4° C. Tissue sections were incubated with Envision+ system HRP-conjugated secondary antibodies for 30 min at RT and labeling was visualized by adding liquid DAB+. Consecutive sections of 3 μm from TNBC breast samples were used to evaluate CD95L expression in CD31 or D2-40 positive endothelial cells. Sections were counterstained (hematoxylin) and mounted with DPX mounting medium. Negative controls for non-specific staining excluded the addition of primary antibody, which was replaced by normal mouse IgG.

CD95L ELISA.

Anti-CD95L ELISA (Diaclone, Besançon, France) was performed following the manufacturer's recommendations to accurately quantify the amount of cleaved-CD95L present in sera.

Flow Cytometry Analysis.

Cells were pre-incubated for 30 min at 37° C. with 3 μM of H2FDA resuspended in PBS. The cells were then pre-incubated for an additional 30 min with or without ROS inhibitors before stimulation with 100 ng/ml cl-CD95L for the indicated times. Cells were then washed and analyzed by FACScalibur (BD Bioscience).

Immunoblot Analysis.

Cells were lysed for 30 min at 4° C. in lysis buffer [25 mM HEPES (pH 7.4), 1% v/v Triton X-100, 150 mM NaCl, and 2 mM EGTA supplemented with a mix of protease inhibitors (Sigma-Aldrich)]. Protein concentration was determined by the bicinchoninic acid method (PIERCE, Rockford, Ill., USA) according to the manufacturer's protocol. Proteins were separated by SDS-PAGE and transferred to a nitrocellulose membrane (GE Healthcare, Buckinghamshire, England). The membrane was blocked for 15 min with TBST (50 mM Tris, 160 mM NaCl, 0.05% v/v Tween 20, pH 7.8) containing 5% w/v dried skimmed milk (TBSTM) and then incubated overnight with primary antibody at 4° C. in TBSTM. The membrane was intensively washed (TBST) and peroxidase-labeled anti-rabbit or anti-mouse (SouthernBiotech, Birmingham, Ala., US) was added for 45 min. The proteins were visualized with the enhanced chemiluminescence substrate kit (ECL, GE Healthcare).

MISC Immunoprecipitation.

Breast cancer cells (20×10⁶ cells per condition) were incubated with 100 ng/ml cl-CD95L for the indicated times. The cells were then lysed, 1 μg of Apo1-3 was added to the cell lysate and CD95 was immunoprecipitated by the addition of protein A-sepharose beads (Sigma-Aldrich). For the immunoprecipitation of EGF-R, 1 μg mouse anti-EGF-R (Santa Cruz Biotechnology, CA, USA) was added to the cell lysate and EGF-R was immunoprecipitated with protein A-sepharose beads (Sigma-Aldrich). After extensive washing, the immune complex was resolved by SDS-PAGE.

In Vitro Motility Assays.

Boyden chambers (Millipore, Molsheim, France) containing 8 μm pore membranes were cultured in 24-well plates. After hydration of the membranes, 10⁵ cells were added to the top chamber in low serum (1%)-containing medium. The bottom chamber was filled with low serum (1%)-containing medium in the presence or absence of 100 ng/ml cl-CD95L. Cells were cultured for 24 h at 37° C. in a 5% CO2 humidified incubator. To quantify invasion, cells were fixed with methanol and stained with Giemsa. Stained cells were then removed from the top side of the membrane using a cotton-tipped swab and five representative pictures for each insert were taken of the invading cells from the reverse side. For each experiment, cells were lysed and absorbance at 560 nm was measured.

Immunofluorescence Imaging.

Cells were allowed to adhere for 24 hours on cover slips before being treated with 100 ng/ml cl-CD95L for the indicated times at 37° C. After extensive washing, cells were fixed for 15 min in PBS containing 4% w/v paraformaldehyde. The aldehyde groups were quenched for 10 min in PBS supplemented with 5% FCS. The fixed cells were then incubated with 5 μg/ml anti-CD95 mAb (DX2) for 30 min at 4° C. Finally, CD95 was visualized using Alexa488-conjugated goat anti-mouse antibody (Invitrogen) diluted in PBS for 30 min at 4° C. EGF-R was stained by incubating the cells with 2 μg/ml Alexa555-conjugated anti-EGF-R mAb (Millipore) for 30 min at 4° C. Nuclei were stained with the far-red fluorescent DNA dye DRAQ5™ (Cell Signaling Technology, Boston, Mass., USA). Cells were washed with PBS, dried, and mounted with Fluorescent Mounting Media (Dako, Carpinteria, Calif., USA). Images were acquired using a TSC SP5 confocal microscope (Leica, Wetzlar, Germany) with a 63× objective.

Ca²⁺ Monitoring.

For experiments on parent cell lines, breast cancer cells were loaded with Fura2-PE3-AM (1 μM) at room temperature for 30 min in Hank's Balanced Salt Solution (HBSS). After washing with HBSS, the cells were incubated for 15 min in the absence of Fura2-AM to complete de-esterification of the dye. Cells were placed in the temperature controlled chamber (37° C.) of an inverted epifluorescence microscope (Olympus IX70) equipped with an ×40 UApo/340-1.15 W water-immersion objective (Olympus), and fluorescence micrograph images were captured at 510 nm and 12-bit resolution by a fast-scan camera (CoolSNAP fx Monochrome, Photometrics). To minimize UV light exposure, a 4×4 binning function was used. Fura2-AM was alternately excited at 340 and 380 nm, and the ratios of the resulting images (excitations at 340 and 380 nm and emission filter at 520 nm) were produced at constant intervals (10 seconds). The Fura-2 ratio (Fᵣₐₜᵢₒ 340/380) images were displayed and the F-ratio values from regions of interest (ROIs) drawn on individual cells were monitored during the experiments and analyzed offline with Universal Imaging software, including Metafluor and Metamorph. Fᵣₐₜᵢₒ reflects the intracellular Ca²⁺ concentration changes. Each experiment was repeated 3 times, and for each experimental condition, the average of more than 20 single-cell traces was used.

For experiments on GFP-expressing cell lines, Fluo8-AM was used, instead of Fura2-PE3-AM for experiments with GFP-expressing cells (infected with shRNA-expressing lentivirus), because GFP fluorescence disturbs Ca²⁺ measurement with Fura2-PE3. Efficiently transduced cells (GFP expressing cells) were located by their emission of fluorescence at 530±30 nm for a light excitation at 485±22 nm. Ca²⁺ changes were evaluated by exciting Fluo8-AM-loaded cells at 535±35 nm. The values of the emitted fluorescence (605±50 nm) for each cell (F) were normalized to the starting fluorescence (F₀) and reported as F/F₀ (relative Ca²⁺_{[CYT]}). Only GFP-positive cells were considered. As for Fura2-PE3-AM, T cells were loaded with Fluo8-AM (1 μM) for 30 min in Hank's Balanced Salt Solution (HBSS) and then incubated for 15 min in the Fura2-AM free HBSS to complete de-esterification of the dye.

Results

Serum CD95L Levels Predict Metastasis in Women with TNBC.

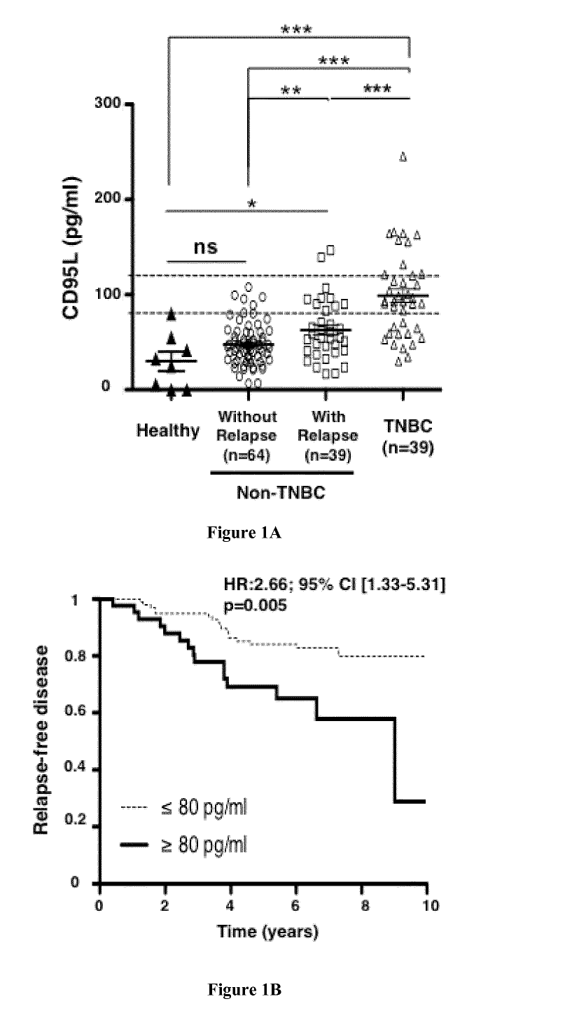

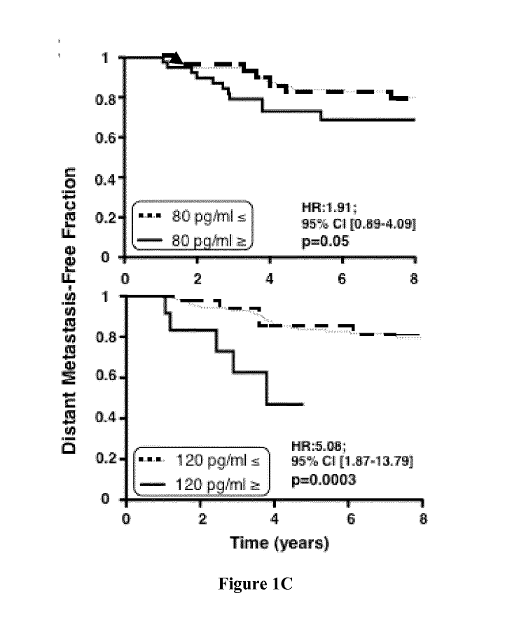

Although high amounts of CD95L have been detected in breast cancer tissue and associated with pathology progression (16), the biological role of this ligand in carcinogenesis remains unknown. According to our data showing that CD95L, released in serum after shedding by metalloproteases, is endowed with a pro-motile effect (11), we wondered if the serum concentration of CD95L was increased in women with breast cancer and associated with risk of metastasis. In this regard, the levels of CD95L in the serum of TNBC, non-TNBC patients and women affected by benign mammary hyperplasia (healthy subjects) were quantified. Patients with TNBC exhibited an increased amount of serum CD95L compared to patients affected by non-TNBC (mean at 98.94±45.37, n=39 vs 52.79±26.2, n=103, P<0.0001) and subjects with benign breast diseases (98.94±45.37, n=39 vs 30.04±28.52, n=8, P<0.0001, FIG. 1A). Of note, the quantity of CD95L in patients affected by non-TNBC displayed a heterogeneous distribution (FIG. 1A); however, when this cohort of patients was sub-divided based on the occurrence of relapse, CD95L levels were significantly higher in relapsing patients (mean at 62.72±31.31, n=35 vs 47.68±21.77, n=68, P=0.0053, FIG. 1A). Kaplan-Meier analyses showed that both TNBC and non-TNBC patients with CD95L concentrations higher than or equal to 80 pg/ml exhibited significantly reduced disease-free survival (FIG. 1B) and an increased occurrence of metastases (FIG. 1C). This predisposition to metastasis was even more pronounced in patients with concentrations of serum CD95L higher than or equal to 120 pg/ml (FIG. 1C).

High amounts of CD95L in breast cancers raise the question of what are the cells producing this transmembrane ligand. To address this point, CD95L expression was evaluated by immunohistochemistry on breast tissue sections from healthy, non-triple-negative and TNBC patients. While CD95L staining was barely detectable in healthy subjects, its expression level was enhanced in non-TNBC and TNBC patients. When correlated with serum levels, CD95L expression displayed an ascending expression gradient from healthy subjects to TNBC patients. Of note, CD95L expression was observed in endothelial cells covering tumor blood vessels (CD31⁺) surrounding small tumor masses but not in the lymphatic endothelium (D2-40⁺; (17)). Put together, these findings pinpoint that serum CD95L is a bad prognostic factor for risk of relapse and distant metastasis in breast cancers and is primarily produced by blood vessels surrounding breast cancer cells.

Cleaved CD95L Induces a p110β-Driven Pro-Motile Signal in TNBC Cells

Next, the capacity of soluble CD95L cleaved by metalloprotease (cl-CD95L) to promote migration in breast cancer cells was investigated. To produce purified cl-CD95L, we transfected the epithelial kidney cells 293 with wild type CD95L-encoding cDNA. These cells also secreted exosomes, which contained full length CD95L, and thus contaminated supernatants. To eliminate this contaminant, supernatants underwent an ultracentrifugation step to pellet the secreted vesicles (8). Cl-CD95L remained in exosome-free supernatant at a lower molecular weight (≈30 KDa) than the transmembrane ligand (≈40 kDa) (data not shown and (11)). Then, TNBC breast cancer cell lines (Hs578T, MDA-MB-231 and MDA-MB-468) were exposed to cl-CD95L and cell migration was monitored using Boyden chambers. All TNBC cells stimulated with cl-CD95L exhibited a dramatic increase in motility. Of note, exposure to levels of CD95L equal to those measured in breast cancer patients was sufficient to trigger cell motility. Indeed, titration of cl-CD95L showed that cl-CD95L at 100 pg/ml, a dose corresponding to mean concentration measured in TNBC patients (98.94±45.37 pg/ml vs. 30.04±28.52 pg/ml in healthy donors, induced migration of TNBC cells.

Class I PI3Ks are comprised of four catalytic isoforms (α, β, δ and γ) whose lipid kinase activity generates the second messenger PIP3, which ultimately serves as a docking site for various signaling factors. For instance, binding of the serine-threonine kinase Akt to PIP3 via its pleckstrin homology domain leads to its redistribution to the plasma membrane, where it undergoes phosphorylation by PDK-1 at Thr³⁰⁸ and by mTOR complex-2 at Ser⁴⁷³ (For review, see (18)). Activation of the PI3K/Akt signaling pathway is reported to promote different steps leading to metastases including cell motility and intravasation/extravasation (19). Therefore, we wondered if this signal was triggered in breast malignant cells exposed to cl-CD95L. In all tested TNBC cell lines, CD95L induced rapid phosphorylation of Akt at Ser⁴⁷³, a hallmark of PI3K activation. Furthermore, inhibition of the PI3K signal using a non-cytotoxic concentration of the pharmacologic inhibitor LY294002 abrogated the migration of TNBC cells exposed to cl-CD95L, indicating that metalloprotease-processed CD95L acts as a chemoattractant for malignant breast cells through activation of PI3K. The class I PI3K catalytic isoforms p110 α and β are ubiquitously expressed, while p110δ and γ expression are restricted to hematologic cells. To investigate the contribution of p110α and β to CD95-mediated cell motility, RNA interference was used to silence their expression. Whereas down-regulation of p110α did not alter CD95-mediated PI3K activation and cell migration, silencing of p110β abrogated Akt phosphorylation and cell motility. These findings demonstrated that cl-CD95L selectively activates the PI3K catalytic subunit p110β, which promotes cell migration of TNBC cells.

Cl-CD95L Triggers MISC Formation in TNBC Cells.

In the presence of cl-CD95L, T lymphocytes undergo the formation of a receptosome termed MISC, containing the src kinase c-yes (11). To evaluate whether this complex also forms in TNBC cells, MDA-MB-231 and MDA-MB-468 cells were stimulated with cl-CD95L followed by immunoprecipitation of CD95. Analysis of the resulting immune complex revealed that CD95 did not bind the DISC components FADD and caspase-8 but did recruit c-yes. In addition, infection of TNBC cells with lentivirus expressing c-yes-targeting shRNA down-modulated the expression level of c-yes and prevented both Akt phosphorylation and cell migration in TNBC cells exposed to cl-CD95L. These data indicated that cl-CD95L induces MISC formation in breast cancer cells.

Because calcium (Ca²⁺) plays a pivotal role in cell motility (20), its impact in non-orthodox signaling induced by cl-CD95 in breast cancer cells was analyzed. In non-excitable cells, store-operated calcium (SOC) influx is the major mechanism of Ca²⁺ entry (21, 22), and recent studies identified Orai1 as a pore-forming component of the SOC channel (23, 24). Pre-treatment of breast cancer cells with the Ca²⁺ chelator, BAPTA-AM, or the pharmacological inhibitor of SOC channels, BTP2 (25) inhibited both CD95-mediated PI3K activation and cell migration. To next determine whether Orai1 participated in CD95 signaling in breast cancer cells, Orai1 expression was suppressed by shRNA. Down-regulation of Orai1 did not alter the initial step of the CD95-mediated Ca²⁺ response, its mobilization from the endoplasmic reticulum, but it abolished the subsequent Ca²⁺ entry observed in TNBC cells stimulated with cl-CD95L. Of note, Orai1-driven Ca²⁺ entry was essential for inducing PI3K activation and cell migration. To further characterize the mechanistic link between CD95 engagement by cl-CD95 and Ca²⁺ signaling in breast tumor cells, the contribution of c-yes and p110β to the CD95-mediated Ca²⁺ response was studied. Silencing of c-yes abrogated CD95-mediated Ca²⁺ signaling. On the other hand, although Ca²⁺ signaling contributed to PI3K activation, the opposite was not true since p110β knock-down did not affect the CD95-mediated Ca²⁺ response. These findings bring to light that c-yes occupies a proximal position in the molecular events leading to activation of PI3K/Ca²⁺ signaling pathway and cell migration of breast tumor cells.

CD95 Recruits EGFR into MISC.

Using the Eukaryotic Linear Motif database (26), an in silico analysis of short linear motifs in the intracellular region of CD95 did not reveal any consensus sequences to explain the recruitment of p110β or its regulatory subunit p85, suggesting that at least a third factor connects CD95 to PI3K signaling. Approximately 72% of TNBC cells express EGFR (ErbB1) (27), expression of which has been correlated with cell migration (28) and EGFR is a potent inducer of the PI3K signaling pathway (29). Accordingly, we wondered whether EGFR contributed to non-orthodox CD95 signaling. EGFR expression was expressed predominantly in TNBC cell lines (MDA-MB-231, MDA-MB-468 and Hs578T) compared to non-TNBC cells (MCF7, T47D and ZR-75-1). The src kinase family has been reported to phosphorylate EGFR at Tyr845, a modification that stabilizes the activation loop and maintains the receptor in an active state (30). Moreover, c-src can also associate with EGFR to form a heterocomplex in TNBC cells (31). Based on our immunoprecipitation experiments revealing an interaction between CD95 and c-yes, it was hypothesized that recruitment of c-yes to MISC may orchestrate the activation of EGFR, which in turn may serve as molecular platform to elicit the PI3K (p110β) signaling pathway. To explore this hypothesis, the phosphorylation status of EGFR at Tyr845 was examined. Exposure of MDA-MB-468 breast cancer cells to cl-CD95L resulted in phosphorylation of EGFR at Tyr845, which reached a peak at 2 min and disappeared after 10 min of stimulation. This EGFR phosphorylation preceded Akt activation. In addition, EGFR phosphorylation was dependent on c-yes, since no EGFR phosphorylation was observed in TNBC cells in which c-yes expression was suppressed. To next analyze whether EGFR was recruited in MISC formed in breast cancer cells exposed to cl-CD95L, biochemical and imaging approaches were carried out. Immunoprecipitation of CD95 or EGFR from breast tumor cells exposed to cl-CD95L revealed the rapid formation of an immune complex containing CD95/c-yes/EGFR/p110β. Confirming these biochemical observations, EGFR and CD95 were co-localized at the leading edge of emitted pseudopodia in MDA-MB-231 and Hs578T TNBC cells. Together, these findings demonstrated that, in the presence of cl-CD95L, an unconventional receptosome encompassing CD95, c-yes, EGFR and p110β forms in breast tumor cells.

EGFR is Essential for Cl-CD95L-Mediated Migration of Breast Cancer Cells.

To further investigate the biological role of EGFR in the CD95 signaling pathway, the activity of EGFR was inhibited with erlotinib. Of note, the clinical effect of this synthetic compound is under investigation in the treatment of metastatic breast cancers previously treated with anthracycline or taxane (see http://clinicaltrials.gov/). Erlotinib prevented CD95-mediated phosphorylation of Akt at serine 473 in both MDA-MB-231 and MDA-MB-468 cells, suggesting that EGFR activity contributes to CD95-mediated PI3K activation in breast tumor cells. In addition, a non-cytotoxic amount of erlotinib abolished the migration of TNBC cells exposed to cl-CD95L. To demonstrate the role of EGFR in CD95-mediated cell signaling, we next silenced this receptor tyrosine kinase using shRNA targeting different regions in the EGFR mRNA. Down-modulation of EGFR expression inhibited both CD95-mediated PI3K activation and cell migration in TNBC cells. Next, the role of EGFR in the cl-CD95L-induced Ca²⁺ response was examined. Surprisingly, although the binding of EGF to EGFR evoked a Ca²⁺ response, which is blocked by erlotinib in MDA-MB-231 cells, neither treatment with erlotinib nor EGFR silencing altered the CD95-mediated Ca²⁺ response. This latter observation questioned the implication of EGF in the recruitment and activation of EGFR in breast cancer cells stimulated with cl-CD95L. To address whether or not EGF was involved in this process, cl-CD95L-mediated signaling was examined in TNBC cells pre-incubated with cetuximab, an antibody that binds to EGFR ectodomain and prevents its interaction with EGF (32). Strikingly, high concentrations of this blocking antibody, which abrogated the EGF-induced Ca²⁺ response, did not alter CD95-mediated activation of PI3K and cell motility, supporting the notion that CD95-driven recruitment of EGFR occurs through an EGF-independent mechanism in TNBC cells.

ROS Production Initiates the CD95-Mediated Non-Apoptotic Signal.

Based on our findings, activation of c-yes corresponds to the most proximal event in the non-orthodox signaling pathway triggered by cl-CD95L in TNBC cells. Since recent reports have shown that src kinases can behave as redox sensors promoting cell migration in leukocytes (33), we next wondered whether CD95 in TNBC cells activated c-yes through the generation of reactive oxygen species (ROS). Breast cancer cells exposed to cl-CD95L experienced a rapid increase in intracellular ROS, which was blocked by pre-treatment with reduced nicotinamide adenine dinucleotide phosphate oxidase (NADPHox) inhibitors, such as diphenyleneiodonium chloride (DPI) and apocynin (34). To address the impact of ROS in c-yes activation, we evaluated the activation status of c-yes by following its auto-phosphorylation at Tyrosine 426. Importantly, pre-treatment of mammary cancer cells with non-cytotoxic amounts of DPI or apocynin impaired CD95-mediated activation of c-yes and Aid and inhibited cell migration. The Nox family is defined by seven distinct transmembrane catalytic subunits that form the basis of the enzyme, Nox-1 to -5 and Duox-1 and Duox-2. While Nox-1 to -4 are associated with p22^{Phox}, which is involved in their proper membrane targeting and activity, Nox-5, Duox-1 and Duox-2 do not require p22^{phox} for their activity (35). Analysis of MISC revealed the recruitment of p22^{Phox}. In addition, MDA-MB-231 and MDA-MB-468 TNBC cells in which p22^{Phox} expression has been down-modulated using shRNA-encoding lentivirus failed to induce c-yes and Akt phosphorylation and did not migrate when exposed to cl-CD95L suggesting that association of p22^{Phox} with Nox1, 2, 3 or 4 may cause the CD95-mediated ROS production. We next investigated the Nox sub-unit recruited in MISC formed in TNBC cells encountering cl-CD95L. While Nox-1, -2 and -4 were not detected associated with CD95 in MDA-MB-231 stimulated with cl-CD95L, Nox3 was rapidly recruited. Furthermore, Nox3 down-modulation prevented PI3K activation and cell migration in TNBC cells stimulated with cl-CD95L.

These findings disclose that in TNBC cells, interaction of CD95 with cl-CD95L triggers a rapid NADPHox-driven ROS production causing activation of c-yes and its downstream signaling pathway. Of note, inhibition of NADPH oxidase completely abrogated the CD95-mediated Ca²⁺ response, confirming that CD95-triggered ROS generation is a proximal event in the signaling cascade induced by metalloprotease-cleaved CD95L in TNBC cells.

### Discussion

This study highlights that an elevated serum concentration of CD95L in women diagnosed with breast cancer is associated with poor prognosis and strong risk of distant metastasis. Strikingly, CD95L is expressed primarily by the endothelium on blood vessels surrounding the breast tumor mass and is not detected on endothelial cells covering lymphatic vessels. This distribution of CD95L in the tumor architecture suggests that its shedding by a yet unknown metalloprotease may create the concentration gradient required to promote intravasation of breast tumor cells and thus distant cancer metastases. Also, multivariate analysis does not reveal correlation between concentration of CD95L (higher than or equal to 80 pg/ml or to 120 pg/ml) and lymph-node positive disease supporting that while CD95L could promote passage of malignant cells through blood vessels leading to distant metastases (hematogenous), a CD95L-independent mechanism controls regional spread of tumor cells (lymphogenic).

Further analysis of the molecular pathway connecting the “death receptor” CD95 to downstream PI3K signaling in TNBC cells exposed to cl-CD95L revealed the recruitment and activation of EGFR (ErbB1). Surprisingly, although EGFR is mandatory for CD95-mediated activation of PI3K, it is dispensable for the Ca²⁺ response, indicating that the two pathways diverge earlier in the unconventional cl-CD95L-triggered signaling. This divergence occurs downstream of ROS generation and subsequent c-yes activation and upstream of EGFR activation, since inhibition of NADPH oxidase and silencing of c-yes expression abolishes both PI3K activation and Ca²⁺ signaling in TNBC cells exposed to cl-CD95L. In presence of EGF, TNBC cells evoke a Ca²⁺ response that is abolished by pre-treatment with tyrosine kinase inhibitor Erlotinib. Given that Erlotinib and EGFR down-regulation do not alter the Ca²⁺ signal observed in breast cancer cells exposed to cl-CD95L, we surmise that recruitment and activation of EGFR by CD95 occurs through an atypical mechanism independent of EGF. Confirming this assumption, the EGFR neutralizing antibody cetuximab does not impair the CD95-driven EGFR activation in breast cancer cells. Conventionally, binding of EGF ligands to EGFR leads to receptor dimerization, activation of its intrinsic tyrosine kinase activity and subsequent phosphorylation of downstream signaling molecules (36). However, this dogma has been challenged by the discovery that the activation of EGFR-mediated signaling can occur in a ligand-independent manner in the presence of ROS (37). In addition, G protein-coupled receptor activation can mediate EGFR transactivation through the activation of Src family tyrosine kinases (38), and c-Src itself is able to facilitate EGFR activation by phosphorylation of Tyr⁸⁴⁵ (31). Our results bring to light a novel mechanism for EGFR activation in TNBC cells stimulated with metalloprotease-cleaved CD95L. From a mechanistic standpoint, CD95-driven EGFR activation relies on the generation of ROS by Nox3 activating c-yes, which in turn recruits and activates EGFR. Similarly to EGFR, c-Met is a receptor tyrosine kinase for hepatocyte growth factor (HGF) (39). It is worth noting that the CD95 signaling pathway can be modulated by the transmembrane receptor c-Met, which sequesters the death receptor and hampers the induction of the apoptotic signaling pathway in hepatocytes (40). Amino-acid residues YLGA in c-Met interact with CD95 (40) and disrupt its homotrimeric self-aggregation. Importantly, this minimal amino-acid sequence, which is also found in CD95L, is not detected in EGFR sequence. In addition, while binding of cl-CD95L to CD95 is instrumental in recruiting EGFR, CD95L disrupts the CD95/c-Met association (41). Put together, these findings strongly suggest that receptor tyrosine kinases such as c-Met and EGFR can interact with CD95 by different molecular mechanisms. More importantly, these results bring to light that implementation of CD95-mediated non-apoptotic signals may occur through the recruitment of tyrosine kinase receptors that behave as docking sites for several proteins including PI3K and PLCγ.

Because the level of serum CD95L is associated with relapse and distant metastasis in breast cancers, exhaustive identification of the components of MISC and characterization of the molecular pathway leading to the induction of CD95-mediated non-apoptotic signaling is crucial to understand how this “death receptor” can transmit non-death signals depending on the ligand with which it interacts. Our findings point to the activation of the src kinase c-yes, which implements Orai1-mediated store-operated calcium (Ca²) entry (SOCE), recruits EGFR, and induces the downstream activation of the p110β catalytic isoform of PI3K. Ca²⁺ responses mainly occur in a biphasic manner resulting from the activation of IP₃ receptors and the release of Ca²⁺ from the endoplasmic reticulum (ER) followed by sustained SOCE across the plasma membrane (42). In TNBC cells, Orai1 is a major contributor to SOCE (43), which plays a pivotal role in both the replenishment of ER stores and cell signaling (44). Of note, although silencing of Orai1 expression did not alter the initial Ca²⁺ mobilization from the ER stores, it completely abrogated CD95-mediated motility in TNBC cells, bringing to light that SOCE is instrumental in cl-CD95L-mediated breast cancer cell migration.

Many PI3K inhibitors have been tested in clinical trials at different stages (45). Although PI3K inhibitor treatment regimens are relatively well-tolerated by patients, it is important to determine the specific isoforms involved in different biological processes since their clinical targeting may result in undesirable side effects, such hyperglycemia related to the pivotal role of p110α in insulin signaling (45). Some phase I/II clinical trials are currently set to examine the selective inhibition of PI3K p110α and β isoforms for the treatment of breast cancer metastasis. Based on our data, we predict that selective inhibition of the PI3Kβ iso form may be more effective for the treatment of TNBC and prevention of metastasis.

In summary, this study identifies serum CD95L levels as a new prognostic marker of metastatic dissemination in women with breast cancer. This finding may help to guide clinicians in selection of the most appropriate treatment regimen for patients with high levels of cl-CD95L who should undergo strong therapeutic treatments while the ones showing low concentration should be spared. In addition, this atypical CD95-mediated signaling presents new therapeutic targets for preventing metastatic dissemination in TNBC. Although inhibitors of EGFR kinase activity may be attractive, especially since gefitinib and erlotinib are already used for the treatment of non-small cell lung cancer, most patients on prolonged gefitinib and erlotinib treatment develop secondary mutations in the EGFR kinase domain that block drug binding, leading to clinical resistance (Kobayashi et al., 2005; Kwak et al., 2005; Pao et al., 2005). Therefore, inhibition of the CD95/CD95L interaction may be a more appropriate treatment approach, since such an inhibitor already exists and is well-tolerated by patients (46).

### REFERENCES

Throughout this application, various references describe the state of the art to which this invention pertains. The disclosures of these references are hereby incorporated by reference into the present disclosure.
1. Christofori G (2006) New signals from the invasive front. Nature 441(7092):444-450.
2. Sotiriou C & Pusztai L (2009) Gene-expression signatures in breast cancer. N Engl J Med 360(8):790-800.
3. Perou C M, et al. (2000) Molecular portraits of human breast tumours. Nature 406(6797):747-752.
4. Davies C, et al. (2011) Relevance of breast cancer hormone receptors and other factors to the efficacy of adjuvant tamoxifen: patient-level meta-analysis of randomised trials. Lancet 378(9793):771-784.
5. Dowsett M, et al. (2010) Meta-analysis of breast cancer outcomes in adjuvant trials of aromatase inhibitors versus tamoxifen. Journal of clinical oncology: official journal of the American Society of Clinical Oncology 28(3):509-518.
6. Peto R, et al. (2012) Comparisons between different polychemotherapy regimens for early breast cancer: meta-analyses of long-term outcome among 100,000 women in 123 randomised trials. Lancet 379(9814):432-444.
7. Tauzin S, Debure L, Moreau J F, & Legembre P (2012) CD95-mediated cell signaling in cancer: mutations and post-translational modulations. Cellular and molecular life sciences: CMLS 69(8): 1261-1277.
8. Schneider P, et al. (1998) Conversion of membrane-bound Fas(CD95) ligand to its soluble form is associated with downregulation of its proapoptotic activity and loss of liver toxicity. J Exp Med 187(8): 1205-1213.
9. Suda T, Hashimoto H, Tanaka M, Ochi T, & Nagata S (1997) Membrane Fas ligand kills human peripheral blood T lymphocytes, and soluble Fas ligand blocks the killing. J Exp Med 186(12):2045-2050.
10. O'Reilly L A, et al. (2009) Membrane-bound Fas ligand only is essential for Fas-induced apoptosis. Nature 461(7264):659-663.
11. Tauzin S, et al. (2011) The naturally processed CD95L elicits a c-yes/calcium/PI3K-driven cell migration pathway. PLoS Biol 9(6):e1001090.
12. Chen L, et al. (2010) CD95 promotes tumour growth. Nature 465(7297):492-496.
13. Kleber S, et al. (2008) Yes and PI3K bind CD95 to signal invasion of glioblastoma. Cancer Cell 13(3):235-248.
14. Bivona T G, et al. (2011) FAS and NF-kappaB signalling modulate dependence of lung cancers on mutant EGFR. Nature 471(7339):523-526.
15. Kischkel F C, et al. (1995) Cytotoxicity-dependent APO-1 (Fas/CD95)-associated proteins form a death-inducing signaling complex (DISC) with the receptor. Embo J 14(22):5579-5588.
16. Reimer T, et al. (2000) FasL:Fas ratio—a prognostic factor in breast carcinomas. Cancer Res 60(4):822-828.
17. Kahn H J & Marks A (2002) A new monoclonal antibody, D2-40, for detection of lymphatic invasion in primary tumors. Lab Invest 82(9):1255-1257.
18. Vanhaesebroeck B, Stephens L, & Hawkins P (2012) PI3K signalling: the path to discovery and understanding. Nat Rev Mol Cell Biol 13(3):195-203.
19. Qiao M, Sheng S, & Pardee A B (2008) Metastasis and AKT activation. Cell Cycle 7(19):2991-2996.
20. Evans J H & Falke J J (2007) Ca2+ influx is an essential component of the positive-feedback loop that maintains leading-edge structure and activity in macrophages. Proc Natl Acad Sci USA 104(41):16176-16181.
21. Lewis R S (2007) The molecular choreography of a store-operated calcium channel. Nature 446(7133):284-287.
22. Parekh A B & Putney J W, Jr. (2005) Store-operated calcium channels. Physiol Rev 85(2):757-810.
23. Prakriya M, et al. (2006) Orai1 is an essential pore subunit of the CRAC channel. Nature 443(7108):230-233.
24. Yeromin A V, et al. (2006) Molecular identification of the CRAC channel by altered ion selectivity in a mutant of Orai. Nature 443(7108):226-229.
25. Zitt C, et al. (2004) Potent inhibition of Ca2+ release-activated Ca2+ channels and T-lymphocyte activation by the pyrazole derivative BTP2. J Biol Chem 279(13):12427-12437.
26. Dinkel H, et al. (2012) ELM—the database of eukaryotic linear motifs. Nucleic Acids Res 40(Database issue):D242-251.
27. Carey L, Winer E, Viale G, Cameron D, & Gianni L (2010) Triple-negative breast cancer: disease entity or title of convenience? Nature reviews. Clinical oncology 7(12):683-692.
28. Nie F, et al. (2012) Involvement of epidermal growth factor receptor overexpression in the promotion of breast cancer brain metastasis. Cancer.
29. Holgado-Madruga M, Emlet D R, Moscatello D K, Godwin A K, & Wong A J (1996) A Grb2-associated docking protein in EGF- and insulin-receptor signalling. Nature 379(6565):560-564.
30. Cooper J A & Howell B (1993) The when and how of Src regulation. Cell 73(6): 1051-1054.
31. Biscardi J S, et al. (1999) c-Src-mediated phosphorylation of the epidermal growth factor receptor on Tyr845 and Tyr1101 is associated with modulation of receptor function. J Biol Chem 274(12):8335-8343.
32. Mano M & Humblet Y (2008) Drug Insight: panitumumab, a human EGFR-targeted monoclonal antibody with promising clinical activity in colorectal cancer. Nat Clin Pract Oncol 5(7):415-425.
33. Yoo S K, Starnes T W, Deng Q, & Huttenlocher A (2011) Lyn is a redox sensor that mediates leukocyte wound attraction in vivo. Nature 480(7375):109-112.
34. Bedard K & Krause K H (2007) The NOX family of ROS-generating NADPH oxidases: physiology and pathophysiology. Physiol Rev 87(1):245-313.
35. Al Ghouleh I, et al. (2011) Oxidases and peroxidases in cardiovascular and lung disease: new concepts in reactive oxygen species signaling. Free radical biology & medicine 51(7):1271-1288.
36. Cochet C, et al. (1988) Demonstration of epidermal growth factor-induced receptor dimerization in living cells using a chemical covalent cross-linking agent. J Biol Chem 263(7):3290-3295.
37. Filosto S, et al. (2011) EGF receptor exposed to oxidative stress acquires abnormal phosphorylation and aberrant activated conformation that impairs canonical dimerization. PLoS ONE 6(8):e23240.
38. Luttrell L M, Della Rocca G J, van Biesen T, Luttrell D K, & Lefkowitz R J (1997) Gbetagamma subunits mediate Src-dependent phosphorylation of the epidermal growth factor receptor. A scaffold for G protein-coupled receptor-mediated Ras activation. J Biol Chem 272(7):4637-4644.
39. Bottaro D P, et al. (1991) Identification of the hepatocyte growth factor receptor as the c-met proto-oncogene product. Science 251(4995):802-804.
40. Zou C, et al. (2007) Lack of Fas antagonism by Met in human fatty liver disease. Nat Med 13(9): 1078-1085.
41. Wang X, et al. (2002) A mechanism of cell survival: sequestration of Fas by the HGF receptor Met. Mol Cell 9(2):411-421.
42. Oh-hora M & Rao A (2008) Calcium signaling in lymphocytes. Curr Opin Immunol 20(3):250-258.
43. Motiani R K, Abdullaev I F, & Trebak M (2010) A novel native store-operated calcium channel encoded by Orai3: selective requirement of Orai3 versus Orai1 in estrogen receptor-positive versus estrogen receptor-negative breast cancer cells. J Biol Chem 285(25):19173-19183.
44. Qian D & Weiss A (1997) T cell antigen receptor signal transduction. Curr Opin Cell Biol 9(2):205-212.
45. Shuttleworth S J, et al. (2011) Progress in the preclinical discovery and clinical development of class I and dual class I/IV phosphoinositide 3-kinase (PI3K) inhibitors. Current medicinal chemistry 18(18):2686-2714.
46. Tuettenberg J, et al. (2012) Pharmacokinetics, pharmacodynamics, safety and tolerability of APG101, a CD95-Fc fusion protein, in healthy volunteers and two glioma patients. International immunopharmacology 13(1):93-100.

1. A method for predicting the risk of relapse and distant metastasis in a patient suffering from a triple negative breast cancer comprising the steps of
i) determining the level of soluble CD95L in a blood sample obtained from the patient
ii) comparing the level determined at step i) with a predetermined reference value and
iii) concluding that the patient will exhibit an increased risk of relapse and distant metastasis when the level determined at step i) is higher than the predetermined reference value or concluding that the patient will exhibit a decreased risk of relapse and distant metastasis when the level determined at step i) is lower than the predetermined reference value.

2. A method for predicting the disease-free survival in a patient suffering from a triple negative breast cancer comprising the steps of
i) determining the level of soluble CD95L in a blood sample obtained from the patient
ii) comparing the level determined at step i) with a predetermined reference value and
iii) concluding that the patient has a poor prognosis when the level determined at step i) is higher than the predetermined reference value or concluding that the patient has a good prognosis when the level determined at step i) is lower than the predetermined reference value.

3. A kit for performing the method of claim 1, said kit comprising means for measuring the level of soluble CD95L in the blood sample obtained from the patient.

4. The kit of claim 3 wherein said means for measuring the level of soluble CD95L is an antibody that interacts specifically with soluble CD95L.

5. A method for the treatment of triple negative breast cancer in a subject in need thereof comprising administering to the subject a therapeutically effective amount of a CD95 antagonist.

6. A method for the prevention of metastases in a subject suffering from a triple negative breast cancer comprising administering to the subject a therapeutically effective amount of a CD95 antagonist.

7. A method of preventing metastases in a subject suffering from triple negative breast cancer comprising the steps of i) predicting the risk of relapse and distant metastasis by performing the method of claim 1 and ii) administering to the subject a therapeutically effective amount of a CD95 antagonist when it is concluded at step iii) that the subject will exhibit an increased risk of relapse and distant metastasis.

8. The method according to claim 5, wherein the CD95 antagonist is selected from the group consisting of anti-soluble CD95L antibodies and anti-CD95 antibodies.

9. The method according to claim 8 wherein the anti-CD95 antibody binds to the regions delimitated between the amino acid at position 43 and the amino acid at position 66 of CD95 (SEQ ID NO:1).

10. The method according to claim 8 wherein the CD95 antagonist is a fully human fusion protein consisting of the extracellular domain of the CD95 receptor and the Fc domain of an IgG antibody.

11. The method according to claim 5, wherein the CD95 antagonist is an inhibitor of CD95 or CD95L expression.

12. A method of preventing metastases in a subject suffering from triple negative breast cancer comprising the steps consisting of i) predicting the risk of relapse and distant metastasis by the method of claim 1 and ii) administering to the subject a therapeutically effective amount of a EGFR antagonist when it is concluded at step iii) that the level of soluble CD95L is higher than the predetermined reference value and thus that the subject will exhibit an increased risk of relapse and distant metastasis.

13. A method of preventing metastases in a subject suffering from triple negative breast cancer comprising the steps consisting of i) predicting the risk of relapse and distant metastasis by performing the method of claim 1 and ii) administering to the subject a therapeutically effective amount of a PI3K inhibitor when it is concluded at step iii) that the subject will exhibit an increased risk of relapse and distant metastasis.
